(19) United States
(12) Plant Patent Application Publication
(10) Pub. No.: US 2003/0150038 P1
(43) Pub. Date: Aug. 7, 2003

(54) VERBENA PLANT NAMED 'SUNMARIRO'

(76) Inventor: Yasunori Yomo, Yasu-gun (JP)

Correspondence Address: BURNS, DOANE, SWECKER & MATHIS, L.L.P., P.O. Box 1404, Alexandria, VA, 22313-1404, US

(21) Appl. No.: 10/066,734

(22) Filed: Feb. 6, 2002

(51) Int. Cl. A01H 5/00

(52) U.S. Cl. PLT/308

(57) ABSTRACT

Disclosed herein is a new and distinct variety of Verbena plant having a spreading growth habit with medium stems and a very broad plant spreading area. The Verbena plant has abundant branching particularly with respect to primary branching, and a great profusion of blooms, with the entire plant remaining in bloom for a considerable period of time. The flowers are large with petals having a vivid red color without eye color, and a plentiful number of flowers in a spike. The blooming period is from early April to November, and the flowering duration is long. The plant exhibits high tolerance to cold and heat, high resistance to pests and diseases, particularly powdery mildew, and high resistance to rain.

### BACKGROUND OF THE VARIETY

The present invention relates to a new and distinct variety of verbena plant obtained from a crossing of verbena plant variety Verbena hybrida ‘H39-2’ and the same Verbena hybrida ‘H39-2’.

The verbena is a very popular plant and is used for flower bedding and potting in the summer season. There are only a few varieties of the verbena plant which have a spread growth habit with medium stems, much branching, many flowers in a spike and a high resistance to heat, cold, and diseases. Accordingly, this invention is aimed at obtaining a new variety having a spread growth habit, much branching, many flowers in a spike, large diameter flowers, high tolerance to heat and cold, and resistance to diseases and pests, and also having a vivid red color petal.

The verbena plant variety Verbena hybrida ‘H39-2’ was obtained from open-pollination with the verbena plant variety Verbena hybrida ‘135-2’ and an unknown verbena plant. Seedlings were obtained in the July of 1997, from crossing ‘H39-2’ as the female parent and the same verbena plant variety Verbena hybrida ‘H39-2’ as the pollen parent in April of 1997, and were grown from the September of 1997 at the Hakushu Nursery center of SUNTORY Ltd., located at 2913-1, Oaza-Toriharamukoubayasi, Hakushu-cho, Kitakoma-gun, Yamanashi-ken, Japan, and then plants were obtained. Among these plants, one was selected in view of a spread growth habit and petal color, and propagated by cutting, and then grown as a trial growth by flower bedding and planting from January of 1998. Finally, only one plant was selected from plants a trial on which was carried out by flower potting and bedding in January of 1998, and the botanically characteristics of its seedlings were examined, using similar varieties ‘SUNMARIPI’ (Registration. No. 4900 under the Seeds and Seedlings Law of Japan) and ‘SUNMARIRIPI’ (Registration. No. 8146 under the Seeds and Seedlings Law of Japan) for comparison. As a result, it was concluded that this verbena is distinguishable from any other variety whose existence is known, is uniform and stable in its characteristics, and then this new variety of verbena plant was named ‘SUNMARIRO’.

In the following description, the color-coding is in accordance with the Horticultural Color Chart of The Royal Horticultural Society, London, England (R.H.S. Color Chart), and the Inter-Society color Council-Nation Bureau of Standard Color Name (I.S.C.C.-N.B.S. Color Name). A color chart based on The Japan Color Standard for Horticultural Plants (J.H.S. Color Chart) is also added for reference.

The variety ‘H39-2’ used as the female parent and the pollen parent in the obtaining of the new variety ‘SUNMARIRO’ is one of our breed varieties and was not filed in Japan or any other country. Variety ‘H39-2’ was obtained from open-pollination with the verbena plant variety Verbena hybrida ‘135-2’ and an unknown verbena plant. The main botanical characteristics of variety ‘H39-2’ are as follows.

Plant:

Growth habit.—Spreading.

Extension.—Broad; 80-100 cm.

Height.—Medium; 15-20 cm.

Stem:

Diameter.—Medium; 1.5-2.5 mm.

Anthocyanin pigmentation.—Present.

Pubescence.—Medium.

Prickle.—Absent.

Branching.—Abundant.

Subterranean stem.—Absent. But when the stems contact the surface of soil, the nodes take root in the ground and the plant has a spreading growth habit.

Length of internode.—Medium; 2.0-4.0 cm.

Leaf:

Phyllotaxis.—Opposite.

Shape of blade.—Oblong.

Depth of blade incision.—Shallow.

Blade crenation of margin.—Serrate.

Length.—Medium; 3.0-4.5 cm.

Width.—Medium; 1.5-2.5 cm.

Color.—Grayish olive green. (R.H.S. Color Chart No. 137A, JHS Color Chart No. 3716).

Pubescence.—Sparse.

Petiole.—Present.

Diameter of petiole.—Medium; 1.0-1.2 mm.

Length of petiole.—Short; 2.0-2.2 mm.

Flower:

Shape of cluster.—Funnel-shaped.

Length of cluster.—Medium; 30-50 mm.

Diameter of cluster.—Medium; 50-60 mm.

Facing direction.—Upward.

Outward curvature of petal.—Slightly curved.

Diameter.—Large; 1.5-2.0 cm.

Height.—High; 2.0-3.0 cm.

Color.—Vivid red. (R.H.S. Color Chart No. 45B, JHS Color Chart No. 0407).

Eye color.—Absent.

Variegation on petal.—Absent.

Color intensity.—Absent.

Overlapping of petals.—Separate.

Incision of petal.—Present.

Number of petals.—Medium.

Incision of calyx.—Present.

Length of calyx.—Long; 1.0-1.2 cm.

Anthocyanin pigmentation of calyx limb.—Present.

Shape of pistil.—Two lobes.

Number of stamens.—Medium.

Color of anther.—Yellow green.

Diameter of peduncle.—Thin; 1.0-2.0 mm.

Length of Peduncle.—Medium; 40-50 mm.

Number of flowers.—Medium.

Reproductive organs.—1 pistil and 4 stamens.

Flower fragrance.—Absent.

Flowering time.—Medium.

Flowering duration.—Long.

Physiological and ecological characteristics: High resistance to diseases and pests, particularly powdery mildew. High tolerance to heat and drought. High tolerance to rain and to cold.

The variety ‘SUNMARIPI’ used as a comparison for the new variety ‘SUNMARIRO’ is one of our breed varieties and was registered in Japan in 1996 (Registration No. 4900 under the Seeds and Seedlings Law of Japan) and in the US in 1998 (Registration No. Plant 10705 as a plant patent), and so on. The main botanical characteristics of ‘SUNMARIPI’ are as follows.

Plant:

Growth habit.—Spreading.

Plant extension.—Broad. (50-70 cm).

Plant height.—Medium. (15-25 cm).

Stem:

Diameter.—Medium. (2-3 mm).

Anthocyanin pigmentation.—Present.

Pubescence.—Medium.

Prickle.—Absent.

Branching.—Medium.

Subterranean stem.—Absent.

Length of internode.—Medium. (4.0-5.0 cm).

Leaf:

Phyllotaxis.—Opposite.

Shape of blade.—Hastate.

Blade of incision.—Present.

Depth of brade incision.—Shallow.

Crenation of blade margin.—Serrate.

Length.—Medium. (4.0-5.0 cm).

Width.—Medium. (2.0-2.5 cm).

Color.—Grayish olive green. (R.H.S. Color Chart No. 137A, JHS Color Chart No. 3716).

Pubescence.—Medium.

Petiole.—Present.

Petiole diameter.—Medium. (approximately 1.0 mm).

Petiole length.—Medium. (approximately 2.0 mm).

Flower:

Shape of cluster.—Funnel-shaped.

Cluster length.—Medium. (3.0-3.5 cm).

Cluster diameter.—Medium. (5.0-5.5 cm).

Facing direction.—Upward.

Outward curvature of petal.—Curved.

Diameter.—Large. (1.5-2.0 cm).

Height.—Medium. (approximately 2.0 cm).

Color of petal.—Deep purplish pink (R.H.S. Color Chart No. 70C, JHS Color Chart No. 9213).

Eye color.—Absent.

Variegation.—Absent.

Color intensity.—Present.

Overlapping of petals.—Separate.

Number of petal.—Medium; 5.

Calyx incision.—Absent.

Calyx length.—Long. (approximately 1.0 cm).

Anthocyanin pigmentation of calyx limb.—Present.

Number of stamens.—Medium.

Color of anther.—Yellow green.

Peduncle diameter.—Medium. (2.0-3.0 mm).

Peduncle length.—Medium. (5.0-6.0 cm).

Number of flowers.—Many. (14-16).

Reproductive organs.—1 pistil and 4 stamens.

Flower fragrance.—Absent.

Flowering time.—Early.

Flowering duration.—Long.

Physiological and ecological characteristics:

Tolerance to cold.—Medium.

Tolerance to heat.—High.

Resistance to diseases.—High.

Resistance to pests.—High.

The variety ‘SUNMARIRIPI’ used as a comparison for the new variety ‘SUNMARIRO’ are our bred variety and was registered in Japan in 2000 (Registration No. 8146 under the Seeds and Seedlings Law of Japan) and in the US in 1999 (Registration No. Plant 11037 as a plant patent), and so on. The main botanical characteristics of ‘SUNMARIRIPI’ are as follows.

Plant:

Growth habit.—Spreading.

Plant extension.—Broad. (50-70 cm).

Plant height.—Medium. (10-20 cm).

Stem:

Diameter.—Medium. (2.0-3.0 mm).

Anthocyanin pigmentation.—Present.

Pubescence.—Medium.

Prickle.—Absent.

Branching.—Medium.

Subterranean stem.—Absent.

Length of internode.—Medium. (2.0-4.0 cm).

Leaf:

Phyllotaxis.—Opposite.

Shape of blade.—Hastate.

Blade of incision.—Present.

Depth of brade incision.—Shallow.

Crenation of blade margin.—Serrate.

Length.—Medium. (2.5-4.5 cm).

Width.—Medium. (1.5-2.5 cm).

Color.—Grayish olive green. (R.H.S. Color Chart No. 137A, JHS Color Chart No. 3716).

Pubescence.—Sparse.

Petiole.—Present.

Petiole diameter.—Medium.

Petiole length.—Short. (2.0-2.2 mm).

Flower:

Shape of cluster.—Funnel-shaped.

Cluster length.—Medium. (3.0-6.1 cm).

Cluster diameter.—Medium. (5.1-5.6 cm).

Facing direction.—Upward.

Outward curvature of petal.—Slightly curved.

Diameter.—Large. (1.5-2.0 cm).

Height.—Medium. (1.7-2.0 cm).

Color of petal.—Deep purplish pink (R.H.S. Color Chart No. 55A, JHS Color Chart No. 9705).

Eye color.—Present.

Variegation.—Absent.

Color intensity.—Absent.

Overlapping of petals.—Separate.

Number of petals.—Medium. (5).

Calyx incision.—Present.

Calyx length.—Long. (1.1-1.3 cm).

Anthocyanin pigmentation of calyx limb.—Absent.

Peduncle diameter.—Thin. (1.0-2.3 mm).

Peduncle length.—Medium. (2.8-3.2 cm).

Number of flowers.—Many. (14-16).

Reproductive organs.—1 pistil and 4 stamens.

Flower fragrance.—Absent.

Flowering time.—Late.

Flowering duration.—Long.

Physiological and ecological characteristics:

Tolerance to cold.—High.

Tolerance to heat.—High.

Resistance to diseases.—High.

Resistance to pests.—High.

This new variety of verbena plant ‘SUNMARIRO’ was propagated by cutting at the Ohmi R&D center of Suntory Ltd., located at 863-1, Aza-Iketani, Omori-cho, Youkaichi-shi, Shiga-ken, Japan, and the homogeneity and stability thereof were confirmed.

### SUMMARY OF THE VARIETY

This new variety of verbena plant has a spreading growth habit having medium stems, and the spreading area of the plant is very broad. The plant has much branching and a plentiful number of flowers in a spike, and a great profusion of blooms. The blooming period is from early April to November, the flowers remain open in the rainy season and flowering duration is long. And whole bush remains in bloom for a considerable period of time. The flower size is large and the petal color of flowers is vivid red without eye color. The plant has high tolerance to cold and heat, high resistance to pests and diseases, particularly powdery mildew, and high resistance to rain.

### BRIEF DESCRIPTION OF THE DRAWINGS

FIG. 1 is photograph showing a partial view of the new variety of verbena plant planted in the ground;

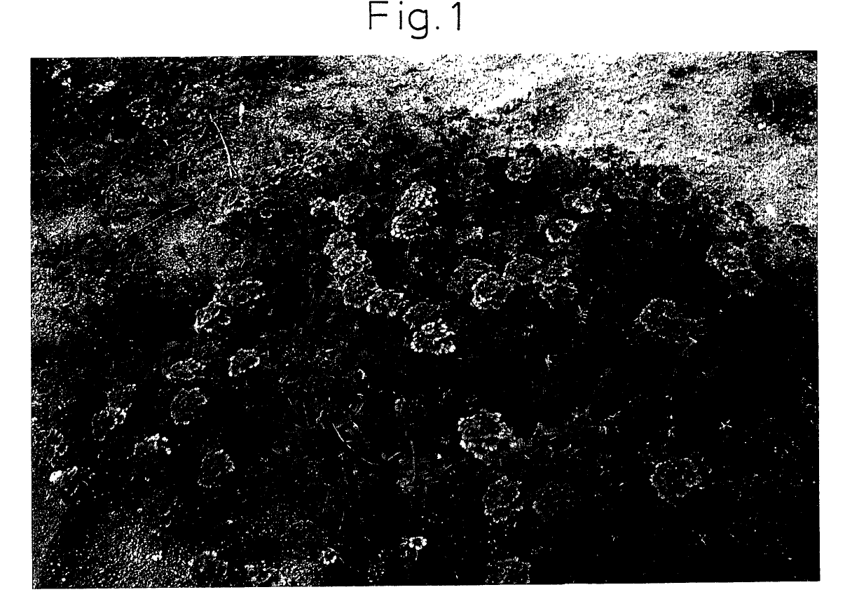

FIG. 2 is a photograph of flowers of the new variety of verbena plant.

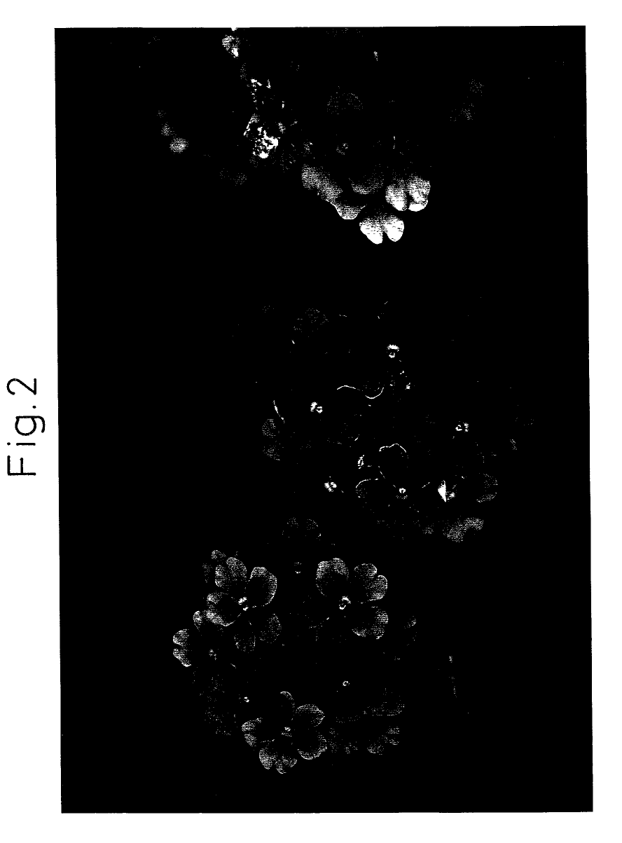

### DESCRIPTION OF THE NEW VARIETY

The botanical characteristics of the new and distinct variety of verbena plant named ‘SUNMARIRO’ are as follows, having observed during August at the Hakushu Nursery center of SUNTORY Ltd., located at 2913-1, Oaza-Toriharamukoubayasi, Hakushu-cho, Kitakoma-gun, Yamanashi-ken, Japan, young plants that had been reproduced by cutting during the preceding May.

Plant:

Growth habit.—Spreading.

Plant extension.—Broad. (100-120 cm).

Plant height.—Medium. (15-18 cm).

Stem:

Diameter.—Medium. (1.5-2.5 mm).

Anthocyanin pigmentation.—Present.

Pubescence.—Medium.

Prickle.—Absent.

Branching.—Many.

Subterranean stem.—Absent.

Length of internode.—Medium (2.5-4.5 cm).

Leaf:

Phyllotaxis.—Opposite.

Shape of blade.—Hastate.

Blade of incision.—Absent.

Depth of brade incision.—Shallow.

Crenation of blade margin.—Serrate.

Length.—Medium. (2.5-3.2 cm).

Width.—Medium. (1.4-2.2 cm).

Color.—Grayish olive green (R.H.S. Color Chart No. 137A, J.H.S. Color Chart No. 3716) on the upper surface and strong yellow green (R.H.S. Color Chart No. 144A and J.H.S. Color Chart No. 3507) on the under surface.

Pubescence.—Sparse.

Petiole.—Present.

Petiole diameter.—Medium. (1.0-1.2 mm).

Petiole length.—Short. (1.5-2.5 mm).

Flower:

Shape of cluster.—Obconical.

Cluster length.—Medium. (42-48 mm).

Cluster diameter.—Medium. (53-60 mm).

Facing direction.—Upward.

Outward curvature of petal.—Slightly curved.

Diameter.—Large. (18-22 mm).

Height.—Medium. (1.7-2.0 cm).

Color of petal.—Single color; Vivid red (R.H.S. Color Chart No. 57A, J.H.S. Color Chart No. 0107) on the upper surface and strong pink (R.H.S. Color Chart No. 58D, J.H.S. Color Chart No. 0104) on the under surface. The petal coloration fades slightly with age.

Eye color.—Absent.

Variegation.—Absent.

Color intensity.—Absent.

Overlapping of petals.—Separate.

Incision of petal.—Present.

Number of petal.—Medium. (5).

Calyx incision.—Present.

Calyx length.—Long. (1.0-1.2 cm).

Anthocyanin pigmentation of calyx limb.—Present.

Pistil shape.—Two lobes.

Stamen number.—Medium.

Anther color.—Yellow green.

Peduncle diameter.—Thin. (1.0-2.0 mm).

Peduncle length.—Medium. (4.0-6.0 cm).

Number of flowers.—Many. (14-16).

Reproductive organs.—1 pistil and 4 stamens.

Flower fragrance.—Absent.

Flowering time.—Early.

Flowering duration.—Long.

Physiological and ecological characteristics:

Tolerance to cold.—High.

Tolerance to heat.—High.

Resistance to diseases.—High.

Resistance to pests.—High.

This new variety of Verbena plant is most suitable for flower bedding and potting, particularly in hanging baskets and containers, and is further excellent for use as ground cover. Pinching of old blossoms will enhance the formation of new blossoms.

The plant of this new variety, ‘SUNMARIRO’, is presently planted and maintained at the Ohmi R&D Center of Suntory Ltd., at 863-1, Aza-Iketani, Omori-cho, Youkaichi-shi, Shiga-ken, Japan.

I claim:

1. A new and distinct variety of Verbena plant, substantially as herein illustrated and described, characterized particularly as to novelty by a spreading growth habit, (B) a plentiful number of flowers in a spike and great profusion of blooms, with the entire plant remaining in bloom for a considerable period of time, (C) a long flowering duration, (D) large flowers and the petals having a vivid red color without eye color, (E) a high resistance to rain, cold, heat, drought, diseases and pests and (F) an early flowering time.
